(19) United States
(12) Patent Application Publication
(10) Pub. No.: US 2008/0188785 A1
(43) Pub. Date: Aug. 7, 2008

(54) Back and muscle support body suit with semi-rigid support rods

(76) Inventors: Kurt D. Shutes, Tucson, AZ (US); Mary B. Shutes, Tucson, AZ (US)

Correspondence Address: Kurt D. Shutes, 1815 S. Olsen Ave, Tucson, AZ, 85713, US

(21) Appl. No.: 12/012,628

(22) Filed: Feb. 4, 2008

Related U.S. Application Data
(60) Provisional application No. 60/899,532, filed on Feb. 5, 2007.

(51) Int. Cl. A61F 5/00; A41D 13/00; A61F 5/02; A41D 1/00

(52) U.S. Cl. 602/19; 2/467; 2/44; 2/69

(57) ABSTRACT

A garment designed to provide support to the major muscle groups of the back and legs in order to reduce muscle fatigue and stress on the vertebrae and their disks. The garment is comprised of several semi-rigid rods (314) that are attached to a lumbar support belt (310) but are free to move vertically within channels of material (410) along the back and the posterior area of the upper thighs. This movement gives the user flexibility and range of motion, both vertically and laterally. The support rods (314) are comprised of a semi-rigid material that will have enough flexibility to bend with the user but enough rigidity to assist the muscles in holding the user's body in a semi-upright position. The garment is comprised of a form fitting material so that the support rods are snug to the body, providing optimum support for the large muscle groups.

### CROSS-REFERENCE TO RELATED APPLICATIONS

This application claims the benefit of PPR Ser. No. 60/899,532 filed Feb. 5, 2007 by the present inventors.

### FEDERALLY SPONSORED RESEARCH

NONE

### SEQUENCE LISTING

NONE

### BACKGROUND OF THE INVENTION

1. Field of Invention

This invention relates to back and muscle supports, specifically to providing support to the muscles of the lower back, backs of the thighs and shoulders while allowing the user freedom of movement.

2. Prior Art

When a person needs to work bent at the waist for extended periods of time, this causes a great deal of stress on the back, thigh and shoulder muscles. Over time, this stress can result in expensive, long term health problems such as disc problems and migraine headaches.

Previous devices intended to address the problem of back fatigue have focused on keeping areas of the spine rigid. Belts that support the lumbar area have been used extensively by companies seeking to avoid costly injuries to their employees. Several patents have been issued trying to improve on these belt devices by using different methods to apply pressure to the lower back or abdomens for example U.S. Pat. No. 5,433,697 (1995), U.S. Pat. No. 5,536,246 (1996), and U.S. Pat. No. 6,932,780 (2005). These devices are only effective for short term support and do not address the other muscle groups of the body that are involved.

Full back braces are designed to provide rigid support such as U.S. Pat. No. 5,256,135 (1993), U.S. Pat. No. 5,571,076 (1996), U.S. Pat. No. 6,478,759 (2002), and U.S. Pat. No. 7,881,348 (2006). Their purpose is to protect the alignment of the spine which does not allow flexibility of movement.

Both braces and belt supports have dealt more with supporting the spine while lifting or keeping the torso immobile rather than assisting the muscles with both lateral as well as vertical movements. Providing sufficient support while still allowing flexibility to the user bridges the gap between the two extremes of total support and minimal, localized support.

3. Objects and advantages

Accordingly, besides the objects and advantages of the support suit described in my above patent, several objects and advantages of the present invention are:

(a) to provide a suit with support rods of varying rigidity in order to assist the muscles in the back, shoulders and thighs and reduce strain in said muscles as well as the spinal and cervical vertebrae;

(b) to provide assistance and support to the muscles of the shoulders, back and thighs that can be increased or decreased according to the needs of the user;

(c) to provide assistance and support to the muscles of the neck, back and thighs that is easy to use and comfortable for extended periods of time;

(d) to provide assistance and support to the muscles of the neck, back and thighs that allows freedom of motion vertically and laterally; and

(e) to provide a suit that provides assistance and support to the muscles of the neck, back and thighs as well as the spinal and cervical vertebrae that is easy to maintain

### SUMMARY

The invention, a full torso support suit, has semi-rigid support rods that are attached to the suit at the lumbar area but are free to move vertically up and down via channels in the material of the suit in order to allow the user to bend and straighten, vertically and laterally, as needed. The suit is made of a form fitting material that keeps the support rods aligned while providing range of motion for the user. The support rods can be removed from the body of the suit which enables the amount of rigidity to be adjusted as well as allow the body of the suit to be easily cleaned.

Accordingly several objects and advantages of the invention are to provide an improved back support, to provide long term support for the muscles of the back as well as the legs and shoulders, to provide support that can be varied as needed by the user, to provide a support that allows freedom of motion, and to provide a support that is easy to maintain. Further objects and advantages will become apparent from a study of the following description and the accompanying drawings.

### DRAWINGS

FIG. 1 is a perspective of the front of the suit.

FIG. 2 is a perspective of the back of the suit without the support rods attached.

FIG. 3 is a perspective of the support rods and attached lumbar belt.

FIG. 4 is a cross-section of the support rods in the channels that allow vertical movement of the rods.

FIG. 5, and FIG. 6 show the same elements of the suit but with the rods as a permanent part of the suit rather than a detachable feature.

### REFERENCE NUMERALS

118 front closure
210 hook and loop attachment
310 lumbar belt
312 lumbar rod attachment
314 semi-rigid support rods
410 material covering support rods
610 padding

### DETAILED DESCRIPTION

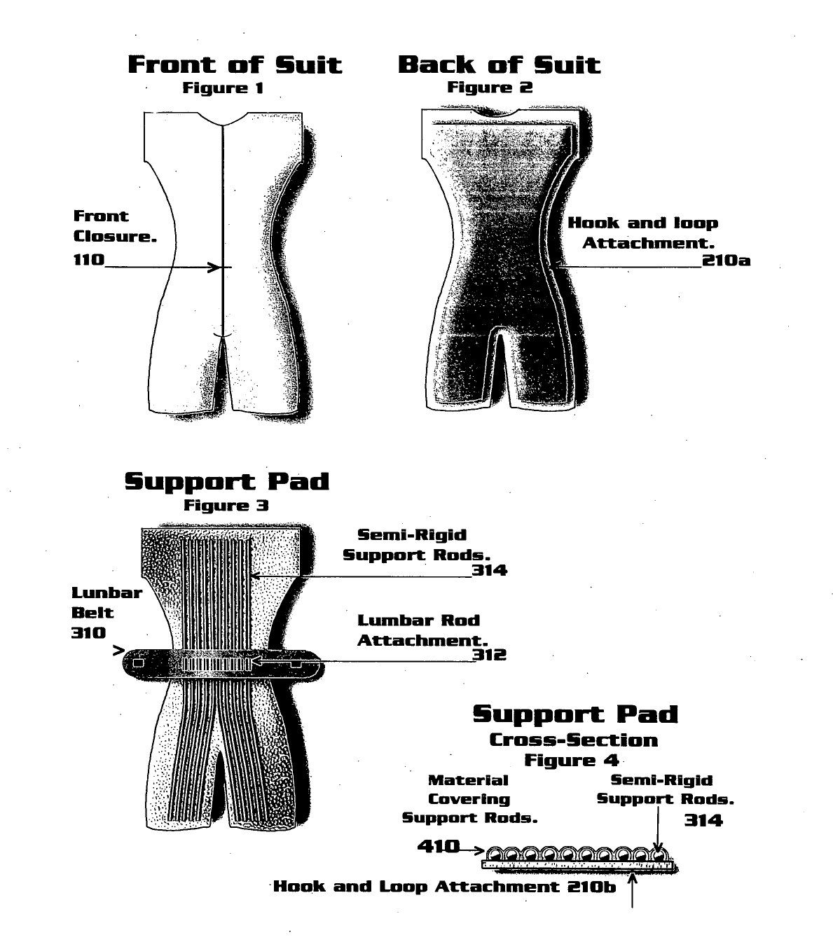

FIG. 1 is a frontal view of the suit. The suit covers the torso from shoulders to just above the knees with a front closure (110) from neck to crotch. The suit itself is made of a formfitting stretchable material such as spandex, preferably a material with properties to wick moisture away from the body to provide comfort to the user. The front closure (111) can be a zipper, snaps, hook and loop closure or any other means of closure.

FIG. 2 is a view of the back of the suit with the detachable support rod pad removed. This shows the hook and loop attachment (210) extending across the shoulders, covering the back of the suit down to the knees. This allows the support rods to be removed from the suit itself, which facilitates the adjustment of the amount of support needed as well as to make the suit easier to clean.

FIG. 3 is a perspective of the detachable support rod pad with a lumbar belt (310). The semi-rigid support rods (314) are encased in channels of fabric (410) down the length of the suit. The support rods are attached to the suit and lumbar belt at the lumbar region (312) but are left open at the shoulders and the knees to allow the rods to move up and down within the channels. The lumbar belt (310) is made of a stretchable material that can be wrapped around to the front and tightly secured to itself. This provides extra lumbar support. The support rods (314) are made of a semi-rigid material that will be rigid enough to provide support to the muscles of the user's torso and thighs but flexible enough to allow freedom of movement, not only vertically but also laterally. These can be made of a variety of materials such as fiberglass or carbon fiber.

FIG. 4 is a cross-section view of the support rods (314) within the fabric channels (410). The fabric channels (410) are sewn to the hook and loop attachment (210) down the entire length of the suit in order to provide a smooth channel for the rods to travel in.

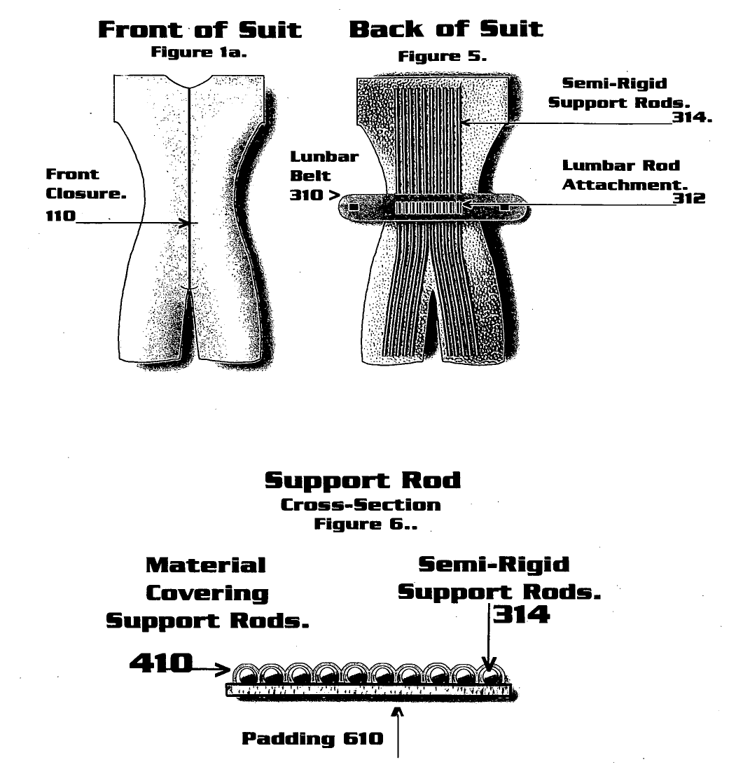

FIG. 5 is a perspective of the back of the suit with the support rods (314) as a permanent feature of the suit. As in FIG. 3, the support rods (314) are attached to the suit and the lumbar belt (310) at the lumbar rod attachment (312) to provide stability. Research and development will provide methods to attach and detach the support rods from the lumbar support to allow adjustment of support as needed by the user. One such mechanism would involve being able to snap the rods into the attachment area due to a ridge on the end of the rod, similar to the mechanism that snaps a spark plug into a motor. The support rods (314) extend the length of the suit from the lumbar belt to the shoulders and the lumbar belt to the knees. The lumbar belt (310) is made of a stretchable material the can be wrapped around to the front and tightly secured to itself to provide extra lumbar support.

FIG. 6 is a cross-section showing the support rods (314) within the fabric channels (410). Between the support rods (314) and the suit there is additional padding (610) for comfort. This is not needed with the detachable support rods because the hook and loop (210) provides the padding.

### Operation

In operation, the front closure (110) in FIG. 1 is designed to aid in donning and removing the one piece garment. A one piece suit is preferred because it would allow the form fitting garment to be snug about the user's body without the possible gaps of two separate garments. The single garment also is preferred because it enables the upper body and lower body support rods (314) to be attached at the lumbar belt (312), although further research and development may find ways to make the garment in two pieces. With the suit snug about the body, the support rods (314) are able to provide added support to the major muscle groups in the torso and legs. This provides benefits to any user who needs to work for extended periods of time bent at the waist while maintaining flexibility and freedom of movement.

The hook and loop attachment (210) in FIG. 2 allows a separate support pad (FIG. 3) comprised of the semi-rigid support rods (314) and attached lumbar belt (310) to be removable from the garment. This enables the user to use different support pads (FIG. 3) that would have varying degrees of rigidity. The garment itself could be easily cleaned in an ordinary washing machine without the support pad (FIG. 3) attached.

The support pad (FIG. 3) is comprised of the support rods (314) and the attached lumbar belt (310) attached to the hook and loop material that complements the hook and loop attachment (210a) on the suit. The support rods (314) are made of a material that is rigid enough to assist the muscles in holding the user's body in a semi-upright position for extended periods of time but is flexible enough to allow the user freedom of movement.

By attaching the support rods (314) at the lumbar belt attachment (312), the support rods (314) are able to move in two separate sections; from the waist to the neck of the garment and from the waist to the knees. With the lumbar belt (310) secured around the waist, this places the greatest support in the lumbar region while still allowing the support rods (314) to move freely vertically and laterally with the user. FIG. 4 shows a cross-section of the support rods (314) with the channels of material (410) on one side and the complementing hook and loop attachment (210b). The material (410) is sewn directly to the hook and loop attachment (210b) to create channels for the support rods (314) to move freely up and down from waist to neck and from waist to knees.

Although being able to remove the support pad has its advantages, there are also applications when it is preferable to have the support rods (314) and lumbar belt (310) an integral part of the garment. FIG. 5 and FIG. 6 show this design. The addition of a pad (610) between the support rods and the garment material is for added comfort to the user and to allow smoother movement within the channels of material (410) covering the support rods (314). Additional research and development can result in means to attach and detach the support rods (314 at the lumbar attachment (312) so that the amount of support can be varied in this version also. One means of attachment would comprise of a ridge on the end of the rod that would snap into a tube at the corresponding lumbar attachment (312).

This results in a light weight garment that assists users who need to work in stressful positions for extended periods of time as well as provides a transitional support for those who are recovering from back injuries and/or surgery.

### Advantages

From the description above, a number of advantages of the support suit become evident;

(a) The user is able to work longer with less muscle fatigue due to the added support of the support rods.

(b) The added support to the large muscle groups results in less stress to the vertebrae and discs.

(c) Because the rods slid freely vertically within the fabric channels of the support pad, this allows lateral and vertical freedom of movement.

(d) The capability to adjust the amount of support allows the user to adjust the suit according to the task at hand.

(e) Being able to easily adjust the amount of support in the suit bridges the gap between total immobility and minimal support for the treatment of patients recovering from back injury and/or surgery.

(f) Because the suit itself is made of a lightweight material designed to wick perspiration away from the user, it can be worn comfortably for long periods of time.

### Conclusion, Ramifications, and Scope

Accordingly, the reader will see that the back and muscle support body suit of this invention can be used to provide support to the muscles of the back, neck and thighs when the user needs to maintain stressful positions for extended periods of time. This reduces muscle fatigue and stress to the vertebrae of the neck and back and allows workers such as neurosurgeons, mechanics and some artists, to name a few examples, to perform more efficiently, work longer, and minimize injuries to the lower back and neck. In addition, the support rods can be made of varying rigidity to address the needs of the individual user. The ability to remove the rods from the suit, either by removing the support pad (FIG. 3) or detaching them at the lumbar rod attachment (312), has several advantages in that
the amount of support needed can be varied according to the task at hand.
the amount of support needed can be varied during the treatment of a patient recovering from back injury and/or surgery. As the patient transitions from immobility to gradually regaining full range of motion, the health care provider can change the support rods to rods of different rigidity thereby changing the amount of support.
the suit without the rods attached is easy to keep clean.

The suit is made of a formfitting stretchable material that keeps the support rods properly aligned to the user's body. Using a lightweight, stretchable material also makes the suit comfortable to use for extended periods of time. The material has the additional property of being able to wick moisture away from the user to provide more comfort during hours of use.

Although the description above contains many specificities, these should not be construed as limiting the scope of the invention but as merely providing illustrations of some of the presently preferred embodiments of the invention. For example, while the invention is designed to assist the muscles in the back, neck and thighs, the same principles of using support rods that are able to move in channels can be used to provide support to wrists, elbows, knees, or any area of the body that can benefit from varying support. The support rods (314) can be made of plastic, carbon fiber, laminate or any material that can made of varying rigidity but able still to be flexible to some degree.

Thus, the scope of the invention should be determined by the appended claims and their legal equivalents, rather than by the examples given.

I claim:

1. A garment comprising a plurality of semi-rigid support rods incased in a formfitting suit, the improvement wherein said support rods are flexible enough to allow freedom of movement while providing support to the user's muscles,

2. The suit in claim 1 wherein said suit is made of a stretchable material designed to fit snugly about the user.

3. The material of claim 2 wherein said material has the property to wick moisture away from the user.

4. The support rods in claim 1 wherein one end of said support rods is attached to said suit and the length of said support rod and its opposite end are allowed to move freely.

5. The attachment in claim 4 wherein said attachment is at a natural pivot point of the user's body such as the waist or a joint.

6. The attachment in claim 5 wherein said attachment provides additional support to said pivot point by means of a non-stretchable fabric that is secured with an elastic material, said elastic material wraps snugly about said pivot point.

7. The support rods in claim 4 wherein said support rods are aligned using channels in said suit, said channels to be in opposite directions perpendicular to said attachment, from the point of attachment to the extremities of said suit.

8. The channels in claim 7 wherein each of said channels encases one of said support rods, said channels to be made of a smooth fabric to allow said support rod to slid freely in a vertical motion within said channel.

9. The support rods in claim 4 wherein said support rods are detachable from said suit as one unit by means of a hook and loop attachment.

10. The hook and loop attachment in claim 9 wherein said hook and loop attachment extends laterally across the posterior side of said suit and vertically to each extremity with one half of said hook and loop attachment a part of said suit and the complimentary half of said hook and loop attachment a separate unit comprised of said support rods in their individual channels.

11. The support rods in claim 4 wherein said support rods are detachable individually from said suit at said pivot point attachment by means of a snap-in mechanism similar to that of a spark plug.

12. The support rods in claim 4 wherein said support rods are attached to said suit as a permanent feature.

13. A method of providing support and assistance to muscles while allowing freedom of movement to the user, comprising;
(a) providing a garment of the type comprising a plurality of support rods made of varying rigidity and flexibility, aligned vertically on said garment and able to move freely from a stationary pivot point, said garment to be formfitting to the user,
(b) providing a means to alter the amount of support provided by said suit by replacing said support rods with ones of different rigidity,
whereby said garment provides a transition of the degree of support provided to the muscles ranging from total support to minimal support.
